SEQUENCE LISTING (1 sequence)

SEQ ID NO: 1 - DNA, 1457 bp, Bacillus stratosphericus
tccctggcccacctttcggcggctggctccataaaggttacctcaccgacttcgggtgttgcaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggctaaaccttgcggtctcgcagccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgtccccgaagggaaagccctatctctagggttgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagtttcccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgcaagcagttactcttgcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtgacagccgaaaccgtctttcatccttgaaccatgcggttcaaggaactatccggtattagctccggtttcccggagttatcccagtcttacaggcaggttacccacgtgttactcacccgtccgccgctaacatccgggagcaagctcccttctgtccgctcgacttgcattattaggcacccgccacgggc

(19) United States
(12) Patent Application Publication
(10) Pub. No.: US 2022/0167628 A1
(43) Pub. Date: Jun. 2, 2022

(54) PHOSPHATE-SOLUBILIZING MICROBIAL AGENT FOR MAIZE, AND PREPARATION METHOD AND USE THEREOF

(71) Applicant: Anhui Agricultural University, Hefei (CN)

(72) Inventors: Yuanyuan Cao, Hefei (CN); Ting Li, Hefei (CN); Wenfeng Ai, Hefei (CN); Quande Li, Hefei (CN); Kangmiao Ou, Hefei (CN); Ke Cai, Hefei (CN); Yuxin Zheng, Hefei (CN); Jiali Hu, Hefei (CN); Ruining Deng, Hefei (CN)

(73) Assignee: Anhui Agricultural University, Hefei (CN)

(21) Appl. No.: 17/412,177

(22) Filed: Aug. 25, 2021

(30) Foreign Application Priority Data
Dec. 2, 2020 (CN) 202011389946.3

(51) Int. Cl. A01N 63/22; A01N 25/02; A01N 61/00; C12P 1/04

(52) U.S. Cl. CPC A01N 63/22; C12P 1/04; A01N 61/00; A01N 25/02

(57) ABSTRACT

A phosphate-solubilizing microbial agent for maize is provided. The phosphate-solubilizing microbial agent is obtained by fermentation of a strain CR50; and the strain CR50 is Bacillus stratosphericus (B. stratosphericus), which was deposited in the China Center for Type Culture Collection (CCTCC) on Nov. 10, 2020, with a deposit number of CCTCC M 2020721. The phosphate-solubilizing microbial agent has the ability to solubilize insoluble inorganic phosphorus and insoluble organic phosphorus such as calcium phosphate and calcium phytate, which can increase an available phosphorus content in soil, significantly promote the growth of maize, and increase a maize yield. The phosphate-solubilizing strain also has the ability to produce indoleacetic acid (IAA) and siderophore, and can undergo an agglutination reaction with maize agglutinin due to maize agglutinin affinity. The strain CR50 can colonize at maize roots for a long time under the mediation of agglutinin and stably exert the growth-promoting effect.

### CROSS REFERENCE TO THE RELATED APPLICATIONS

This application is based upon and claims priority to Chinese Patent Application No. 202011389946.3, filed on Dec. 2, 2020, the entire contents of which are incorporated herein by reference.

### TECHNICAL FIELD

The present disclosure belongs to the technical field of microbial growth promotion, and in particular relates to a phosphate-solubilizing microbial agent for maize, and a preparation method and use thereof.

### BACKGROUND

As one of the principal food crops in China, maize is an important raw material for agricultural and industrial production. Phosphorus is a key nutrient element required for ensuring the excellent characteristics of maize throughout the entire growth cycle. Phosphorus contributes to vital activities of maize such as photosynthesis and respiration to promote the fruit development and root growth of maize plants. A deficiency of phosphorus can lead to weakened photosynthesis, reduced resistance, and delayed flowering of the maize plants, thereby greatly reducing the maize yield.

In agricultural production, a large amount of phosphate fertilizer is applied to the soil to increase the crop yield, but an excessive application amount of phosphate fertilizer can pose various problems. For instance, an excess of calcium-magnesium-phosphate fertilizer will interact with zinc to produce zinc phosphate precipitate, which cannot be utilized by crops, thus making the crops exhibit zinc-deficiency symptoms.

As a raw material for phosphate fertilizer, phosphate rock contains a large amount of heavy metal elements such as cadmium and lead, so overfertilization with phosphate fertilizer can cause soil and water pollution. Additionally, since phosphate rock is a non-renewable resource and about 50 million tons of phosphate fertilizer are consumed globally each year, the growing demand for phosphate fertilizer is depleting its source in a future world.

The total phosphorus in soil has a high content and may account for 0.1% to 0.25% of the soil in weight, but most of total phosphorus is present in the form of insoluble compounds that are difficult to absorb and utilize by plants, and available phosphorus that can be directly absorbed by plants only accounts for 0.5% to 2% of the total phosphorus, which is difficult to meet the demand for increasing the crop yield. In soil there are bacteria capable of solubilizing phosphorus, and these bacteria can convert insoluble phosphorus compounds into available phosphorus that can be utilized by plants, thereby promoting plant growth. There have been many studies on applying phosphate-solubilizing bacteria as a microbial fertilizer to the field to increase the available phosphorus content in the soil and reduce the application amount of phosphate fertilizer. However, the commonly-used phosphate-solubilizing bacteria cannot stably colonize at the maize roots and thus cannot fully exert their growth-promoting effect, which limits the use of phosphate-solubilizing microbial agents. Therefore, it has become an inevitable trend for sustainable agricultural development to develop a phosphate-solubilizing microbial agent that is specifically compatible with maize and can stably colonize, thereby promoting the phosphorus absorption and growth of maize, changing the traditional fertilization mode, and reducing the use of chemical fertilizers.

### SUMMARY

In order to solve the problems in the prior art, one objective of the present disclosure is to provide a phosphate-solubilizing microbial agent for maize.

The phosphate-solubilizing microbial agent for maize is obtained by fermentation of a strain CR50; and the strain CR50 is Bacillus stratosphericus (B. stratosphericus), which was deposited in the China Center for Type Culture Collection (CCTCC) on Nov. 10, 2020, with a deposit number of CCTCC M 2020721.

Preferably, the phosphate-solubilizing microbial agent may be a liquid microbial agent or a solid microbial agent.

Preferably, the phosphate-solubilizing microbial agent may be used in seed dressing, seed coating, and root watering for maize.

Preferably, the liquid microbial agent may be applied at an amount of 5 L to 20 L per mu of a maize field, and the liquid microbial agent may have a viable count of 1×10⁸ to 9×10⁸ CFU.mL⁻¹.

Preferably, the solid microbial agent may be applied at an amount of 5 kg to 20 kg per mu of a maize field, and the solid microbial agent may have a viable count of 2×10⁸ to 8×10⁸ CFU.mL⁻¹.

Another objective of the present disclosure is to provide a preparation method of a liquid phosphate-solubilizing microbial agent for maize, including the following steps:

S61: strain activation: inoculating the strain CR50 to a Luria-Bertani (LB) slant, and culturing at 25° C. to 30° C. for 18 h to 30 h; and

S62: liquid medium culture: washing the strain on the LB slant with 5 mL to 10 mL of NS, inoculating the strain in 80 mL to 120 mL of an LB liquid medium at a volume ratio of 2% to 5%, and culturing on a shaker at 25° C. to 30° C. and 120 r.min⁻¹ to 180 r.min⁻¹ for 24 h to 28 h until a resulting CR50 bacterial solution has a concentration of OD₆₈₀≥0.8 to obtain the liquid microbial agent.

Another objective of the present disclosure is to provide a preparation method of a solid phosphate-solubilizing microbial agent for maize, including the following steps:

S71: strain activation: inoculating the strain CR50 to an LB slant, and culturing at 25° C. to 30° C. for 18 h to 30 h;

S72: liquid medium culture: washing the strain on the LB slant with 5 mL to 10 mL of NS, inoculating the strain in 80 mL to 120 mL of an LB liquid medium at a volume ratio of 2% to 5%, and culturing on a shaker at 2520 C. to 30° C. and 120 r.min⁻¹ to 180 r.min⁻¹ for 24 h to 28 h until a resulting CR50 bacterial solution has a concentration of OD₆₈₀≥0.8 to obtain a liquid microbial agent; and

S73. preparation of the solid microbial agent: adding 0.5 g to 1 g of dry peat per ml of the liquid microbial agent into the liquid microbial agent obtained in S72, thoroughly mixing to obtain a resulting mixture and fermenting the resulting mixture for 1 h to 3 h, and placing a product in a cool place to air-dry until there is no moisture on a surface to obtain the solid microbial agent.

The present disclosure has the following beneficial effects:

1. The phosphate-solubilizing microbial agent of the present disclosure shows an obvious growth-promoting effect for maize, and can significantly increase a phosphorus content in maize leaves and increase a maize yield. Maize agglutinin can be generated in maize roots, which is a protein or glycoprotein from non-immune sources and can bind to polysaccharides on bacterial cell walls. The phosphate-solubilizing strain of the present disclosure has maize agglutinin affinity and can undergo an agglutination reaction with maize agglutinin. The strain CR50 can colonize at maize roots for a long time under the mediation of agglutinin and stably exert the growth-promoting effect.

2. The phosphate-solubilizing strain CR50 used in the present disclosure has the ability to solubilize insoluble inorganic phosphorus and insoluble organic phosphorus such as calcium phosphate and calcium phytate, which can increase an available phosphorus content in soil.

Main mechanism of the strain CR50 to solubilize inorganic phosphorus: 1. The strain secretes organic acids such as acetic acid, malic acid, and citric acid in a metabolic process, which reduce a soil pH and chelate with Fe³⁺, Al³⁺, Ca²⁺, and other ions, thereby solubilizing insoluble inorganic phosphate. 2. CO₂ released by the strain during respiration can reduce a pH of an environment, thereby solubilizing insoluble inorganic phosphate.

Main mechanism of the strain CR50 to degrade organic phosphorus: The strain secretes phytase, phosphatase, and other enzymes to degrade insoluble phosphorus into soluble phosphorus.

3. The fruit is a part with the highest economic value in a maize plant. The phosphate-solubilizing microbial agent of the present disclosure shows an obvious growth-promoting effect for maize, leads to a significant increase in the weight of maize grains, and can increase a maize yield.

4. The phosphate-solubilizing strain CR50 of the present disclosure also has the ability to produce indoleacetic acid (IAA) and siderophore. IAA is an important plant auxin, which can regulate and promote the growth of plant buds, stems, and roots and affect the formation of plant organs. Siderophore is capable of binding to Fe³⁺ and can chelate a small amount of Fe³⁺ around a rhizosphere of a plant to form a chelate complex, which can be absorbed and utilized by the plant. In addition, the chelate complex formed by the siderophore secreted by the growth-promoting bacteria and Fe³⁺ cannot be utilized by other plant pathogenic bacteria, which results in the lack of iron around plant roots and thus prevents the pathogenic bacteria from proliferating in the plant rhizosphere, thereby reducing crop diseases.

5. The liquid microbial agent of the present disclosure is applied at an amount of 5 L/mu to 20 L/mu, and at present, the phosphate fertilizer is applied at an amount of 40 kg/mu to 60 kg/mu in maize culture. In contrast, the application of the phosphate-solubilizing microbial agent provided in the present disclosure can reduce the use of phosphate fertilizer, alleviate soil compaction, and reduce energy consumption, so the phosphate-solubilizing microbial agent has promising application prospects.

### DETAILED DESCRIPTION OF THE EMBODIMENTS

The present disclosure will be further described below through examples.

### Example 1

Strain screening

Maize plants with robust growth were selected, the whole root system with soil was dug out, and large clods and other useless residues were removed. 1 g of rhizosphere soil of each sample was collected and placed in nystatin-containing sterile NS, and cultured on a shaker at 160 r.min⁻¹ for 0.5 h to obtain a rhizosphere soil bacterial suspension. 1 mL of the above bacterial suspension was taken and added to 9 mL of nystatin-containing sterile NS, and serially diluted to concentrations of 10⁻⁷. The bacterial suspension was coated on a nystatin-containing solid medium for phosphate-solubilizing bacteria and cultured at a constant temperature of 28° C. for more than 3 d until a transparent circle appeared, where 3 replicates were set for each concentration. The solid medium for phosphate-solubilizing bacteria had the following formula: glucose: 10 g, K₂HPO₄:2.0 g, ammonium sulfate: 0.5 g, NaCl: 0.3 g, KCl: 0.3 g, MgSO₄.7H₂: 0.3 g, FeSO₄.7H₂O: 0.03 g, MnSO₄.4H₂O: 0.03 g, Ca₃(PO₄)₂: 10 g, agar: 15 g, and distilled water: 1,000 mL. A transparent circle diameter H and a colony diameter C were observed and measured, and a ratio of the two (H/C) was calculated. The larger the H/C value, the stronger the phosphate-solubilizing ability of the strain.

The colonies with transparent circles were picked and inoculated in an LB medium with 50 mg.L⁻¹ nystatin, and streaking purification was conducted multiple times to obtain a pure phosphate-solubilizing strain. A strain with phosphate-solubilizing ability screened out in the LB liquid medium was cultured at a constant temperature for 1 d under shaking until cells were at logarithmic growth phase; a resulting culture was transferred into centrifuge tubes and centrifuged at 3,000 r.min⁻¹ for 10 min; bacteria were collected and added into sterile water, and a resulting mixture was repeatedly pipetted up and down with a pipette to allow uniform dispersion of the bacteria and then centrifuged; and the washing process was repeated 3 times to obtain a bacterial suspension. If the bacterial suspension was turbid, a small amount of sterile water could be added for dilution. 25 μL of the bacterial suspension was added dropwise to the center of a glass slide and mixed with an equal volume of maize agglutinin, the glass slide stood at room temperature for 0.5 h to air-dry, and a reaction was observed under a microscope after staining. The strain undergoing an agglutination reaction was picked out to obtain a maize-compatible phosphate-solubilizing strain. The CR50 used in this application is a maize-compatible phosphate-solubilizing strain obtained by this screening method.

The LB medium had the following formula: peptone: 10 g, yeast powder: 5 g, NaCl: 10 g, agar: 15 g, and distilled water: 1,000 mL.

### Example 2

Determination of the phosphate-solubilizing ability of the strain CR50

A selective-medium and agglutinin dual screening method was used to screen out phosphate-solubilizing strains with specific affinity with maize from the rhizosphere of maize, and the phosphate-solubilizing ability was determined for these strains. The strains were prepared into bacterial suspensions, inoculated into different media for phosphate-solubilizing bacteria, and cultured at 28° C. and 160 r.min⁻¹ for 5 d. The medium had the following formula: glucose: 10 g, K₂HPO₄: 2.0 g, ammonium sulfate: 0.5 g, NaCl: 0.3 g, KCl: 0.3 g, MgSO₄.7H₂O: 0.3 g, FeSO₄.7H₂O: 0.03 g, MnSO₄.4H₂O: 0.03 g, insoluble phosphorus: 10 g, and distilled water: 1,000 mL, pH: 7.2. The Mo—Sb anti-spectrophotometry method was used to determine a soluble phosphorus content in each culture.

The strains were compared for the phosphate-solubilizing ability, and the strain CR50 showed the highest phosphate-solubilizing ability. The ability of the strain CR50 to solubilize insoluble phosphorus was shown in Table 1. It can be seen from Table 1 that the strain CR50 exhibited a prominent solubilizing ability for both calcium phosphate (insoluble inorganic phosphorus) and calcium phytate (insoluble organic phosphorus).

TABLE 1
Ability of the strain CR50 to solubilize insoluble phosphorus

| | Ability to solubilize | Ability to solubilize |
|---|---|---|
| | calcium phosphate | calcium phytate |
| Strain | (mg · L⁻¹) | (mg · L⁻¹) |
| CR50 | 125.28 ± 18.08 | 94.10 ± 12.72 |

### Example 3

Identification of the strain

The morphological observation, the physiological and biochemical tests, and the 16SrRNA gene sequence alignment analysis were conducted to identify the strain CR50.

According to the Manual for Identification of Common Bacterial Systems and Berger's Bacterial Identification Manual, the physiological and biochemical characteristics of the strain CR50 were tested. The morphological test included Gram staining; and the physiological and biochemical tests included a starch hydrolysis test, a catalase test, a VP test, an M-R test, a gelatin liquefaction test, a glucose oxidative fermentation test, and an H₂S production test. Results were shown in Table 2.

The 16SrRNA gene sequence of the strain CR50 was submitted to the NCBI database for BLAST alignment, and in combination with the results of the physiological and biochemical tests, it was determined that the strain CR50 was B. stratosphericus. The 16SrRNA gene sequence of the strain CR50 was shown in the appendix.

TABLE 2
Some physiological and biochemical characteristics of the strain CR50

| | | | | | | Glucose | | |
|---|---|---|---|---|---|---|---|---|
| | Starch | | | | Gelatin | oxidative | H₂S | Gram |
| Strain | hydrolysis | Catalase | VP | M-R | liquefaction | fermentation | production | staining |
| CR50 | + | + | + | − | − | Oxidative | − | + |

### Example 4

Determination of the ability of the strain CR50 to produce IAA and siderophore

The strain CR50 was inoculated into a nitrogen liquid medium at an inoculation amount of 2% and cultured at 28° C. and 160 r.min⁻¹ for 5 d; a resulting culture was centrifuged, a resulting precipitate was removed, and a resulting supernatant was collected and added with a corresponding Sackowski's chromogenic solution at a ratio of 1:2; a resulting mixture reacted for 30 min at 25° C. in the dark, and a nitrogen culture without bacteria was used as a blank control; and the absorbance at 530 nm was determined to calculate an IAA production level of the strain CR50. Preparation of the Sackowski's chromogenic solution: 1 mL of a 0.5 mol.L⁻¹ FeCl₃ solution was added to 50 mL of a 35% HClO₄ solution, and a resulting mixture was well mixed. The nitrogen liquid medium had the following formula: sucrose: 10.0 g, K₂HPO₄: 2.0 g, MgSO₄.7H₂O: 0.5 g, NaCl: 0.1 g, yeast extract: 0.5 g, CaCO₃: 0.5 g, agar: 20 g, and distilled water: 400 mL, pH: 7.0.

The strain CR50 was inoculated into an MKB liquid medium at an inoculation amount of 2% and cultured at 28° C. and 160 r.min⁻¹ for 48 h; a resulting culture was centrifuged, and a resulting supernatant was collected and added with a CAS detection solution at a ratio of 1:1; a resulting mixture was thoroughly mixed and stood for 1 h, and distilled water was used as a control for zero adjustment; and the absorbance (A) at a wavelength of 630 nm was determined. In addition, the CAS detection solution was mixed with an MKB medium without bacteria at a ratio of 1:1, and the absorbance (Ar) at 630 nm was determined. The ability of the strain CR50 to produce siderophore was determined by ultraviolet spectrophotometry. The MKB medium had the following formula: casamino acid: 5.0 g, glycerol: 15 mL, K₂HPO₄: 2.5 g, MgSO₄.7H₂O: 2.5 g, and distilled water: 1,000 mL, pH: 7.2. Preparation of the CAS detection solution: 6 mL of a 10 mmol.L⁻¹ cetyltrimethylammonium bromide (CTAB) solution was added to a 100 mL volumetric flask and slightly diluted with double distilled water (DDW); then 1.5 mL of a 1 mmol.L⁻¹FeCl₃ solution and 7.5 mL of a 2 mmol.L⁻¹ chromeazurol (CAS) solution were mixed and slowly added into the volumetric flask along a glass rod; 4.307 g of anhydrous bisdimethylamine (anhydrous piperazine) was weighed and dissolved in about 30 mL of DDW, and 6.25 mL of 12 mol.L⁻¹ HCl was added to obtain a buffer with a pH of 5.6; and the buffer was transferred to the volumetric flask, and a resulting solution was diluted with DDW to 100 mL for later use.

TABLE 3
Ability of the strain CR50 to produce IAA and siderophore

| | Ability to produce IAA | Ability to produce |
|---|---|---|
| Strain | (mg · L⁻¹) | siderophore |
| CR50 | 9.31 ± 0.46 | ++ |

The ability of the strain CR50 to produce IAA and siderophore was shown in Table 3. It can be seen from Table 3 that the strain CR50 had a strong ability to produce IAA and siderophore, indicating that the strain has the potential to promote plant growth and resist pathogenic bacteria.

### Example 5

Preparation of a microbial agent and a field test of the microbial agent for maize

1. Preparation of the microbial agent

A CR50 strain on an LB slant was washed with 5 ml of NS, inoculated into an LB liquid medium at a volume ratio of 2%, and cultured at 28° C. and 160 r.min⁻¹ for 24 h to obtain a CR50 liquid microbial agent. The microbial agent was diluted with water to OD₆₈₀=0.8 before use.

2. Field test of the microbial agent Two groups were set in the test, including an experimental group applied with the microbial agent and a control group applied with no microbial agent. 10 replicates were set for each group. Maize seeds were sown into the field, and after maize seedling emergence, 20 mL of a diluted microbial agent was applied to the roots. After the maize matured, a plant height, an ear diameter, an ear length, the number of filled grains per ear, an ear weight, a phosphorus content in leaves, and a phosphorus content in soil were determined for the experimental group and the control group. Results were shown in Tables 4 and 5.

TABLE 4
Influence of the CR50 liquid microbial agent on the
agronomic traits and yield of maize

| | Plant | Ear | Ear | Number of filled | Ear |
|---|---|---|---|---|---|
| | height | diameter | length | grains per ear | weight |
| Treatment | (cm) | (cm) | (cm) | (grains) | (g) |
| Control | 178.33 ± | 12.90 ± | 12.40 ± | 364.00 ± | 85.84 ± |
| group | 3.61 | 0.70 | 2.17 | 40.15 | 3.40 |
| Experimental | 218.53 ± | 15.57 ± | 18.63 ± | 464.00 ± | 116.13 ± |
| group | 16.29* | 0.25** | 0.42** | 24.58* | 8.57* |

Notes:
*indicates a significant difference between treatments (p < 0.05), and
**indicates an extremely-significant difference between treatments (p < 0.01).

It can be seen from Table 4 that the plant height, the ear diameter, ear length, the number of filled grains per ear, and the ear weight of the experimental group applied with the CR50 microbial agent increased by 22.54%, 20.70%, 50.24%, 27.47%, and 35.29% respectively compared with that of the control group applied with no microbial agent, where the increase in the plant height, the number of filled grains per ear, and the ear weight reached a significant level, and the increase in the ear diameter and the ear length reached an extremely-significant level.

TABLE 5
Influence of the CR50 liquid microbial agent on the phosphorus
content in maize leaves and the phosphorus content in soil

| | Phosphorus | Available |
|---|---|---|
| | content in | phosphorus |
| | leaves | content in soil |
| Treatment | (g · kg⁻¹) | (mg · kg⁻¹) |
| Control group | 2.74 ± 0.16 | 12.86 ± 0.69 |
| Experimental | 3.23 ± 0.025** | 15.43 ± 0.50** |
| group | | |

Note:
**indicates an extremely-significant difference between treatments (p < 0.01).

It can be seen from Table 5 that the phosphorus content in leaves and the available phosphorus content in soil of the experimental group applied with the CR50 microbial agent increased by 17.88% and 19.98% respectively compared with that of the control group applied with no microbial agent, both reaching an extremely-significant level.

It can be seen from Tables 4 and 5 that the increase in the plant height, the ear diameter, the ear length, the number of filled grains per ear, the ear weight, the phosphorus content in leaves, and the available phosphorus content in soil of the experimental group applied with the CR50 microbial agent reached a significant level compared with that of the control group applied with no microbial agent, indicating that the CR50 liquid microbial agent can release insoluble phosphorus in soil, increase the available phosphorus content in soil, promote the phosphorus absorption of maize, and increase the phosphorus content in leaves, thereby promoting the maize growth and increasing the maize yield.

It should be emphasized that the phosphate-solubilizing microbial agent provided by the present disclosure can be used in combination with other microbial agents or products with growth-promoting effects. Although maize is used as an experimental object for the phosphate-solubilizing microbial agent of the present disclosure, the application of the phosphate-solubilizing microbial agent with strain CR50 on other plants shall also be regarded as an implementation of the present disclosure.

The above are only preferred examples of the present disclosure and are not intended to limit the present disclosure. Although the present disclosure is described in detail with reference to the foregoing examples, a person skilled in the art can still make modifications to the technical solutions described in the foregoing examples, or make equivalent replacement to some technical features. Any modifications, equivalent substitutions, improvements, etc. made within the spirit and scope of the present disclosure should be included within the protection scope of the present disclosure.

What is claimed is:

1. A phosphate-solubilizing microbial agent for maize, wherein
the phosphate-solubilizing microbial agent is obtained by fermentation of a strain CR50; and the strain CR50 is Bacillus stratosphericus deposited in China Center for Type Culture Collection (CCTCC) on Nov. 10, 2020, with a deposit number of CCTCC M 2020721.

2. The phosphate-solubilizing microbial agent according to claim 1, wherein
the phosphate-solubilizing microbial agent is a liquid microbial agent or a solid microbial agent.

3. The phosphate-solubilizing microbial agent according to claim 2, wherein
the phosphate-solubilizing microbial agent is used in seed dressing, seed coating, and root watering for the maize.

4. The phosphate-solubilizing microbial agent according to claim 2, wherein
the liquid microbial agent is applied at an amount of 5 L to 20 L per mu of a maize field, and the liquid microbial agent has a viable count of 1×10⁸ to 9×10⁸ CFU.mL⁻¹.

5. The phosphate-solubilizing microbial agent according to claim 3, wherein
the solid microbial agent is applied at an amount of 5 kg to 20 kg per mu of a maize field, and the solid microbial agent has a viable count of 2×10⁸ to 8×10⁸ CFU.mL⁻¹.

6. A preparation method of the phosphate-solubilizing microbial agent according to claim 2, comprising:
preparing the liquid microbial agent through the following steps:
S61: strain activation: inoculating the strain CR50 to a Luria-Bertani (LB) slant, and culturing the strain CR50 at 25° C. to 30° C. for 18 h to 30 h; and
S62: liquid medium culture: washing the strain CR50 on the LB slant with normal saline (NS), inoculating the strain CR50 in 80 mL to 120 mL of an LB liquid medium at a volume ratio of 2% to 5%, and culturing the strain CR50 on a shaker at 25° C. to 30° C. and 120 r.min⁻¹ to 180 r.min⁻¹ for 24 h to 28 h until a resulting CR50 bacterial solution has a concentration of OD₆₈₀≥0.8 to obtain the liquid microbial agent.

7. A preparation method of the phosphate-solubilizing microbial agent according to claim 2, comprising:
preparing the solid microbial agent through the following steps:
S71: strain activation: inoculating the strain CR50 to an LB slant, and culturing the strain CR50 at 25° C. to 30° C. for 18 h to 30 h;
S72: liquid medium culture: washing the strain CR50 on the LB slant with NS, inoculating the strain CR50 in 80 mL to 120 mL of an LB liquid medium at a volume ratio of 2% to 5%, and culturing the strain CR50 on a shaker at 25° C. to 30° C. and 120 r.min⁻¹ to 180 r.min⁻¹ for 24 h to 28 h until a resulting CR50 bacterial solution has a concentration of OD₆₈₀≥0.8 to obtain the liquid microbial agent; and
S73: preparation of the solid microbial agent: adding 0.5 g to 1 g of dry peat per ml of the liquid microbial agent into the liquid microbial agent obtained in S72, thoroughly mixing the dry peat and the liquid microbial agent to obtain a resulting mixture and fermenting the resulting mixture for 1 h to 3 h to obtain a product, and placing the product in a cool place to air-dry until a surface of the product has no moisture to obtain the solid microbial agent.
